(19) United States
(12) Patent Application Publication
(10) Pub. No.: US 2006/0062753 A1
(43) Pub. Date: Mar. 23, 2006

(54) Polymeric quaternary ammonium salts useful as corrosion inhibitors and biocides

(76) Inventors: Ali Naraghi, Missouri City, TX (US); Nihal Obeyesekere, Houston, TX (US)

Correspondence Address: STREETS & STEELE, 13831 NORTHWEST FREEWAY, SUITE 355, HOUSTON, TX, 77040, US

(21) Appl. No.: 10/943,784

(22) Filed: Sep. 17, 2004

(51) Int. Cl. A61K 31/785

(52) U.S. Cl. 424/78.27

(57) ABSTRACT

A composition useful as a biodegradable corrosion inhibitor and a biocide that comprises a polymeric quaternary ammonium salt prepared by a reaction of a polyepihalohydrin with a tertiary amine, wherein the polyepihalohydrin is prepared by a polymerization reaction of an epihalohydrin in the presence of a monomeric poly alcohol and delivered to the corrosion system in a solvent carrier.

### BACKGROUND OF THE INVENTION

1. Field of the Invention

The present invention relates to corrosion inhibitors and more specifically, to the use of poly-quaternary ammonium salts for use as a biodegradable corrosion inhibitor of metal surfaces and as a biocide.

2. Description of the Related Art

The present invention relates generally to the prevention of metallic surfaces from corrosion and microbiologically influenced corrosion (MIC). It is known that oil and gas formations yield hydrocarbon, brine, organic acids, carbon dioxide, hydrogen sulfide and microorganisms. These are very corrosive environments for metal surfaces that come in contact with these fluids. Therefore, metal pipes, pumps, casings, and other metallic production equipment that comes into contact with these fluids are highly vulnerable to corrosion. This is especially true for pipelines used for transporting petroleum products, usually constructed of steel. The corrosion that occurs in these pipelines may be severe, especially when used to transport fluids at high flow velocities.

In oil industry, many streams that are transported through pipelines include mixtures of brine, oil, and gas that are either in separate phases or in a stable emulsion. As the salt content of the brine component of these mixtures increases, especially above 15% of total dissolved solids, corrosion increases sharply. Not surprisingly, pH also influences the corrosive properties of the streams flowing through the pipeline, with low pH brines tending to be more corrosive. Therefore, any organic acids that are contained in the mixture contribute to the corrosivity of the system. Finally, the pressures and temperatures of the mixture contribute to the corrosivity of the system as well, with higher temperatures and pressures resulting in higher corrosivity.

To protect pipelines and steel equipment that are wetted with these mixtures, such as crude oil, a small amount of corrosion inhibitor may be added to the corrosive system. Corrosion inhibitors for metal include chemical compounds that, when present in small quantities in an aggressive medium, inhibit corrosion by bringing about changes in the surface condition of the metal. In addition, a useful corrosion inhibitor may also act as a biocide to eliminate the microbes contained in the crude or other petroleum mixture that may contribute to corrosion of steel or other metal surfaces.

### SUMMARY OF THE INVENTION

This disclosure is directed to the synthesis of the corrosion inhibitor with strong biocide properties, which is effective to protect piping systems and other metal equipment that are used to transport petroleum products. For example, produced petroleum products containing brine are very corrosive to metallic flow lines.

The composition of the present invention is ultimately soluble in salt water and prevents or reduces corrosion of the metal by disrupting the local electrochemical current. This class of chemicals has low toxicity for marine life when discharged into the ocean, thereby protecting marine life in the vicinity of the discharge.

The present invention includes a composition for use as a biocide and corrosion inhibitor comprising a polymeric quaternary ammonium salt prepared by a reaction of a polyepihalohydrin with a tertiary amine, wherein the polyepihalohydrin is prepared by a polymerization reaction of an epihalohydrin in the presence of a monomeric poly alcohol. The composition further includes a solvent carrier for delivering the polymeric ammonium salt to a corrosion system for treatment.

The polymeric quaternary ammonium salt may be represented as 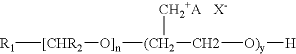
where R₁ and R₂ are organic moieties or H of the poly alcohol R₁(CHOH)ₙR₂, n=1 to 10, y=3 to 150, A is the tertiary amine and X⁻ is a halide. Preferably, n may range between about 3 and about 10 and y may range between about 6 and about 42.

The tertiary amine that is reacted with the polyepihalohydrin may comprise alkyl functional groups. The tertiary amine may further comprise a cycloalkyl functional group or an aryl functional group. Examples of suitable tertiary amines include hexadecyl dimethyl amine, tetradecyl dimethyl amine, dodecyl dimethyl amine, imidazoline or alkyl pyridines.

The polyol may be selected from any primary, secondary or tertiary alcohol such as, for example, glycol, glycerin, any tetritols, any pentitols, sorbitol, any hexitols, mannitol, dulcitol, pentaerythritol, dipentaerythritol, and tripentaerythritol.

The solvent system is preferably comprises components selected from water, methanol, isopropyl alcohol or combinations thereof.

In another embodiment of the present invention, a method of inhibiting corrosion of a metal in contact with a corrosive medium comprises adding a corrosion-inhibiting amount of the composition of claim 1 to the corrosive medium. The corrosive medium may include any type of hydrocarbon or organic stream, with or without water in the stream as, for example, a petroleum product. The petroleum product may be a finished petroleum product, such as diesel, kerosene, NPG, or gasoline or it may be, for example, crude oil. The water making up the corrosive medium may comprise a brine.

The composition may be added in a batch manner, a continuous manner or both. When adding in a batch manner, the dosage rate may be any effective dose, preferably having a range from between about 200 ppm and about 15,000 ppm by volume. When adding in a continuous manner, the dosage rate may be any effective dose, preferably having a range of between about 1 ppm and about 3000 ppm by volume.

### BRIEF DESCRIPTION OF THE DRAWINGS

FIG. 1 illustrates a general reaction for synthesizing the polymeric quaternary ammonium salts useful for the present invention.

FIG. 2 illustrates a general form of a polymeric quaternary ammonium salt of the present invention.

The foregoing and other objects, features and advantages of the invention will be apparent from the following more particular description of a preferred embodiment of the invention, as illustrated in the accompanying drawing.

### DETAILED DESCRIPTION

The present invention provides compositions comprising polymeric quaternary ammonium salts and methods of their use as a corrosion inhibitor and/or biocide. The polymeric quaternary ammonium salts described herein are highly biodegradable, thereby making these compositions highly desirable for use in corrosion systems that require careful consideration concerning environmental impact, such as on offshore drilling platforms. The results of standard testing procedures used for determining biodegradability of these polymeric quaternary ammonium salts demonstrated that these salts were about 20% biodegraded after 7 days, about 95% biodegraded after 14 days and about 97% biodegraded after 28 days. For purposes of comparison, sodium benzoate was about 85.5% biodegraded after 7 days, about 95% biodegraded after 14 days and 100% biodegraded after 28 days.

The polymeric quaternary ammonium salts used in the practice of this invention are prepared by first catalytically polymerizing an epihalohydrin in the presence of an alcohol monomeric compound having the general formula R₁(CHOH)ₙ R₂ where n is between 1 and about 10 and R₁ and R₂ are selected from an alkyl group, H or CH₂OH. In this first step, the reaction proceeds to form an alcohol-epihalohydrin polymer mixture that typically has a polymer length of about 6-42 molecular size. In a second step, the alcohol-epihalohydrin polymer is reacted with tertiary amines to form the polymeric quaternary ammonium salts. A preferred epihalohydrin suitable for use is epichlorohydrin.

The tertiary amines, which are organic compounds that may be considered to be derived from ammonia by replacement of all three hydrogens by functional groups, may be represented in one form by the formula 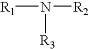
where R₁, R₂ and R₃ may or may not be the same and are a substituted group, preferably a hydrocarbon group such as, for example, alkyl, cycloalkyl, aryl, alkenyl, alkynyl, heterocyclic and substituted derivatives of these. Alkyl groups include, for example, methyl, ethyl, propyl, butyl, amyl, hexyl, heptyl, octyl, nonyl, decyl, undecyl, dodecyl, tridecyl, eicosyl, docosyl and other similar alkyl groups having from between 1 and about 50 or more carbons, preferably between about 1 and about 30 carbons and more preferably between about 1 and 20 carbons. The term “alkyl” also includes isomers of the straight chain group, wherein branching occurs along the chain.

Alkenyl and alkynyl groups include unsaturated analogues of the alkyl groups that contain one or more double or triple carbon-carbon bond such as, for example, decenyl, dodecenyl, tridecenyl, tetradecyl, pentadecenyl, hexadecyl, heptadecenyl, octadecenyl, octadienyl, octatrienyl, alkinyl and butynyl. The terms alkenyl and alkynyl also include isomers of the straight chain group, wherein branching occurs along the chain.

Cycloalkyl groups are saturated ring compounds that include, for example, cyclopentyl, cyclohexyl, cycloheptyl, cyclooctyl and similar and further include substituted derivatives thereof such as, for example alkyl cyclohexyl and dialkyl cyclohexyl groups.

Aryl groups are organic moieties derived from an aromatic compound by removal of one hydrogen and include, for example, phenyl, substituted phenyl, alkyl phenyl, polyalkylphenyl, chlorophenyl, alkoxyphenyl, naphthyl, alkyl naphthyl, benzyl and substituted derivatives of these.

Examples of tertiary amines include, but are not limited to, trimethyl amine, triethyl amine, dimethyl octyl, dimethyl dodecyl, dimethyl tetradecyl, diethyl hexadecyl, methyl ethyl octadecyl, dimethyl octadecyl, dimethyl octadecenyl, diethyl hexadecenyl, dodacylbenzyl methyl, decyl dibenzyl, dimethyl furyl, dimethyl phenyl, diethyl naphthyl, dicyclohexyl methyl and dimethyl cyclohexyl amines.

The R groups of the tertiary amine may also be joined to form cyclic amines such as, for example, morpholines and piperidines and substituted derivatives such as N-alkyl morpholines and N-alkyl piperidines and imidazolines.

In certain instances, two of the R groups are joined to form a cyclic group and the third R becomes a double bond, for example pyridine, alpha-, beta-, or gamma-picoline, other alkyl substituted pyridines, aryl substituted pyridines, alkaryl substituted pyridines, carboxy substituted pyridines, carbalkoxy substituted pyridines, nitro substituted pyridines, alkyloxy substituted pyridines, aryloxy substituted pyridines, acylaminopyridines, alkylaminopyridines, acyl substituted pyridines, and in fact any substituted pyridine. Also there may be used quinoline, isoquinoline, acridine, as well as substituted quinolines, isoquinolines, and acridines in which the substituents are as indicated for the pyridines and indeed, any cyclic compound having one or more tertiary nitrogen atoms.

The alcohol monomer may be any primary, secondary or tertiary alcohol and is preferably a polyol such as, but are not limited to, glycol, glycerin, any tetritols, any pentitols, sorbitol, any hexitols, mannitol, dulcitol, pentaerythritol, dipentaerythritol, and tripentaerythritol. A preferred epihalohydrin is epichlorohydrin. The disclosed alcohols, epichlorohydrin and tertiary amines are commercially available and the usual commercial grades are suitable for the practice of this invention.

The biodegradable characteristic of these polymeric quaternary ammonium salts is increased dramatically by increasing the number of OH groups associated with the polyol. Therefore, preferred polyols of the form R₁(CHOH)ₙR₂ that are useful for synthesizing these salts are those having a higher n value. Preferred values of n are between about 3 and about 10.

A Lewis acid catalyst is used to catalyze the polymerization of the epihalohydrin in the presence of the alcohol monomeric compounds. There are many suitable Lewis acids useful for the required catalyst as known to those having ordinary skill in the art. A preferred catalyst is boron trifluoride etherate.

In general, the polymeric quaternary ammonium salts are prepared by reacting epichlorohydrin, or other epihalohydrin, with a catalytic amount of the Lewis acid. A preferred temperature range for this first step reaction is between about 160° F. and about 180° F. Preferably, the temperature is controlled to stay in within this preferred range. During the second step reaction with the tertiary amine, the temperature range of the reaction is preferably maintained between about 220° F. and about 290° F. The tertiary amine is added to the alcohol-epihalohydrin polymer mixture in a suitable solvent such as, for example, isopropyl alcohol, methanol, and/or ethylene glycol monobutyl ether. The resulting polymeric quaternary ammonium salts are water-soluble and can be diluted with water to form an aqueous solution that is useful as a corrosion inhibitor and biocide.

The polymeric quaternary ammonium salts synthesized as described above have the general form 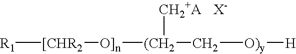
where R₁ and R₂ are the organic moieties or H of the polyol R₁(CHOH)ₙR₂, n is between 1 and about 10, y is between about 3 and about 150, A is the tertiary amine and X⁻ is a halide, preferably chloride.

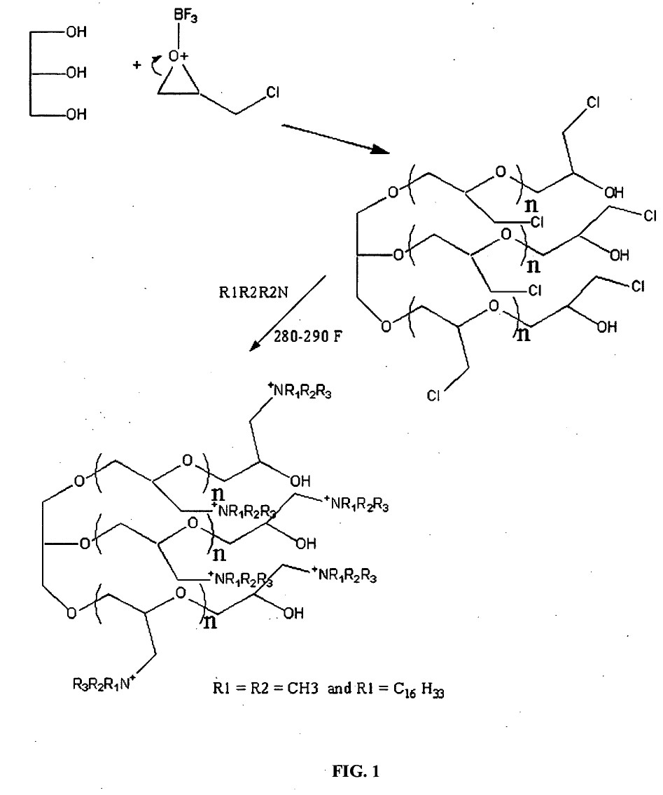

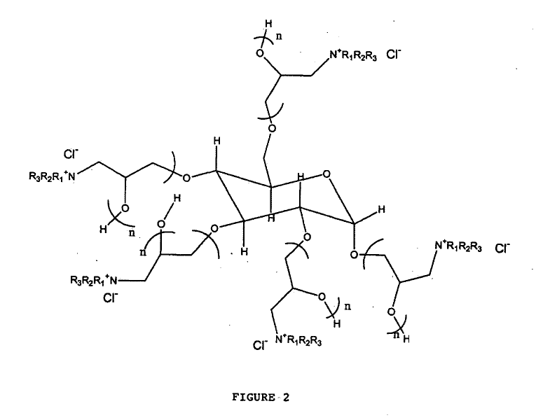

FIG. 1 illustrates a general reaction for synthesizing the polymeric quaternary ammonium salts useful for the present invention. Epichlorohydrin is polymerized in the presence of the monomeric polyol and a Lewis acid catalyst, such as boron trifluoride etherate, to form the polyol-epichlorohydrin polymer. This polymer is then reacted with a tertiary amine, at a temperature of between about 280° F. and about 290° F. to form the polymeric quaternary ammonium salt product. FIG. 2 illustrates a general form of a polymeric quaternary ammonium salt of the present invention formed from a glucose-epichlorohydrin polymer that was reacted with a tertiary amine.

Generally the polymeric quaternary ammonium salts disclosed herein are useful in aqueous/hydrocarbon systems to prevent corrosion of iron-containing metals, such as steel pipelines. These compounds are useful as corrosion inhibitors because they disrupt the local electrochemical current by coating the metal surfaces. Additionally, these compounds reduce or eliminate microbiologically influenced corrosion by killing microorganisms that cause such corrosion such as, for example, sulfur reducing bacteria that can cause pitting in iron-containing metal surfaces. These polymeric quaternary ammonium salts may be used to protect many types of metallic alloys and they are especially useful for protecting mild steel pipelines and equipment. An advantage of these compounds is their biodegradable nature, making these polymeric quaternary ammonium salts desirable because they are so environmentally friendly.

The polymeric quaternary ammonium salts disclosed herein may be used for batch treating or for continuous treating of a hydrocarbon and/or aqueous stream. For continuous treatment of a stream, the dosage is generally effective between about 1 and about 3000 ppm of the total stream, by volume. Preferably, the treatment dosage is between about 1 and about 500 ppm by volume. Preferably, for highly corrosive systems, the treatment dosage is between about 500 and about 2000 ppm by volume. For batch treatment, an effective dosage rate is between about 200 and about 15,000 ppm by volume, and preferably, between about 500 and 10,000 ppm by volume.

The polymeric quaternary ammonium salts disclosed herein may be used alone in a preferred solvent system or they may be used as blends with other chemicals important for chemical effectiveness in a given corrosion inhibitor-treated system. Examples of other effective chemicals that might be delivered in an additive blend include scale inhibitors and paraffin or hydrate inhibitors. A preferred solvent system comprises components selected from water, methanol, isopropyl alcohol or combinations thereof.

The invention will be better understood with reference to the following Examples. It is understood, however, that the Examples are presented only for purposes of illustration and not of limitation. Examples 1-15 provide examples of procedures used to synthesize the polymeric quaternary ammonium salts. The epichlorohydrin used in these examples was the commercially available reagent grade material having a purity of more than 98%. The amines used were commercially available having purities of not less than 98%.

### EXAMPLE 1

This example synthesized the polymeric quaternary ammonium salts from hexadecyl dimethyl amine. First, glycerin-epichlorohydrin polymer was prepared by placing 1.0 mole of glycerin and a catalytic amount of boron trifluoride etherate into a four-necked flask that was fitted with a condenser, a nitrogen sparge tube, a stirrer and a thermometer. Epichlorohydrin (15.0 mol, 1387 g) was placed in an adding funnel.

The flask was heated 160° F. while adding the epichlorohydrin from the funnel. Epichlorohydrin was added drop-wise and the temperature was maintained between about 165° F. and about 180° F. After all the epichlorohydrin was added, the mixture was stirred for one hour at 165° F.

The glycerin-epichlorohydrin polymer (95 g) was added to a kettle containing 100 mL of ethylene glycol monobutyl ether. The polymer was heated to about 220° F. and then 1 mole (267 g) of hexadecyl dimethyl amine was added to the kettle. The mixture was then heated and maintained at a temperature between 270° F. and 290° F. for 17 hours to yield the polymeric quaternary ammonium salt product.

The total amine value of the chemical is a good indicator for the completion of the reaction. The total amine value was less than 0.1%.

### EXAMPLE 2

This example synthesized the polymeric quaternary ammonium salts from tetradecyl dimethyl amine. First, glycerin-epichlorohydrin polymer was prepared as described in Example 1. The polymer (92 g) was added to a kettle containing 100 mL of ethylene glycol monobutyl ether. The polymer was heated to about 220° F. and then 1 mole (239 g) of tetradecyl dimethyl amine was added to the kettle. The mixture was then heated and maintained at a temperature between 270° F. and 280° F. for 17 hours to yield the polymeric quaternary ammonium salt product.

The total amine value of the chemical is a good indicator for the completion of the reaction. The total amine value was less than 0.1%.

### EXAMPLE 3

This example synthesized the polymeric quaternary ammonium salts from dodecyl dimethyl amine. First, glycerin-epichlorohydrin polymer was prepared as described in Example 1. The polymer (92 g) was added to a kettle containing 100 mL of ethylene glycol monobutyl ether. The polymer was heated to about 220° F. and then 1 mole (221 g) of dodecyl dimethyl amine was added to the kettle. The mixture was then heated and maintained at a temperature between 270° F. and 280° F. for 17 hours to yield the polymeric quaternary ammonium salt product.

The total amine value of the chemical is a good indicator for the completion of the reaction. The total amine value was less than 0.1%.

### EXAMPLE 4

This example synthesized the polymeric quaternary ammonium salts from octadecyl dimethyl amine. First, glycerin-epichlorohydrin polymer was prepared as described in Example 1. The polymer (92 g) was added to a kettle containing 100 mL of ethylene glycol monobutyl ether. The polymer was heated to about 220° F. and then 1 mole (297 g) of octadecyl dimethyl amine was added to the kettle. The mixture was then heated and maintained at a temperature between 270° F. and 280° F. for 17 hours to yield the polymeric quaternary ammonium salt product.

### EXAMPLE 5

This example synthesized the polymeric quaternary ammonium salts from amino ethyl ethanol amine, TOFA imidazoline and Alkyl pyridine. First, glycerin-epichlorohydrin polymer was prepared as described in Example 1. The polymer (92 g) was added to a kettle containing 100 mL of ethylene glycol monobutyl ether. The polymer was heated to about 220° F. and then 0.5 mole (175 g) of tall oil fatty acid condensate product (TOFA/AEEA imidazoline) and 0.5 mole (86 g) of alkyl pyridine were added to the kettle. The mixture was then heated and maintained at a temperature between 280° F. and 290° F. for 17 hours to yield the polymeric quaternary ammonium salt product.

### EXAMPLE 6

This example synthesized the polymeric quaternary ammonium salts from octadecyl dimethyl amine. First, glycerin-epichlorohydrin polymer was prepared as described in Example 1. The polymer (92 g) was added to a kettle containing 100 mL of ethylene glycol monobutyl ether. The polymer was heater to about 220° F. and then 0.5 mole (175 g) of TOFA/AEEA imidazoline condensate and 0.5 mole (148.5 g) of octadecyl dimethyl amine was added to the kettle. The mixture was then heated and maintained at a temperature between 280° F. and 290° F. for 17 hours to yield the polymeric quaternary ammonium salt product.

### EXAMPLE 7

This example synthesized the polymeric quaternary ammonium salts from alkyl dimethyl amine. First, glycerin-epichlorohydrin polymer was prepared as described in Example 1. The polymer (92 g) was added to a kettle containing 100 mL of isopropyl alcohol. The polymer was heated to about 220° F. and then 1 mole (296 g) of a mixed alkyl dimethyl amine (alkyl chains are mixture of C-12, C-14 and C-16) was added to the kettle. The mixture was then heated and maintained at a temperature between 280° F. and 290° F. for 17 hours to yield the polymeric quaternary ammonium salt product.

### EXAMPLE 8

This example synthesized the polymeric quaternary ammonium salts from a mixture of alkyl pyridines (C-1 and C-5 alkyl branch). First, glycerin-epichlorohydrin polymer was prepared as described in Example 1. The polymer (92 g) was added to a kettle containing 100 mL of ethylene glycol monobutyl ether and heated to about 220° F. Then, 1.0 mole (166 g) of the mixture of alkyl pyridines was added to the kettle. The mixture was then heated and maintained at a temperature between 280° F. and 290° F. for 17 hours to yield the polymeric quaternary ammonium salt product.

### EXAMPLE 9

This example synthesizes the polymeric quaternary ammonium salts from imidazoline condensed with 4 moles of ethylene oxide. First, glycerin-epichlorohydrin polymer was prepared as described in Example 1. The polymer (92 g) was added to a kettle containing 200 mL of ethylene glycol monobutyl ether. The polymer was heater to about 220° F. and then 1 mole (526 g) of imidazoline condensed with 4 moles of ethylene oxide was added to the kettle. The mixture was then heated and maintained at a temperature of 350° F. for 17 hours to yield the polymeric quaternary ammonium salt product.

### EXAMPLE 10

This example synthesizes the polymeric quaternary ammonium salts from imidazoline condensed with 3 moles of ethylene oxide. First, glycerin-epichlorohydrin polymer was prepared as described in Example 1. The polymer (92 g) was added to a kettle containing 200 mL of ethylene glycol monobutyl ether. The polymer was heater to about 220° F. and then 1 mole (482 g) of imidazoline condensed with three moles of ethylene oxide was added to the kettle. The mixture was then heated and maintained at a temperature of 350° F. for 14 hours to yield the polymeric quaternary ammonium salt product.

### EXAMPLE 11

Glycol-epichlorohydrin polymer was prepared according to the procedure in Example 1 by replacing the glycerin used in Example 1 with glycol. It should be recognized that any polyol may be used in place of glycol or glycerin to form the polyol-epichlorohydrin polymer.

### EXAMPLE 12

One mole of glycol-epichlorohydrin polymer was prepared according to the procedure of Example 11. The procedure of Example 2 was then followed by substituting the glycerin-epichlorohydrin polymer used in Example 2 with the glycol-epichlorohydrin polymer of Example 11. Following the procedure described in Example 2, one mole of tetradecyl dimethyl amine was reacted with the glycol-epichlorohydrin polymer to yield the polymeric quaternary ammonium salt product.

### EXAMPLE 13

One mole of glycol-epichlorohydrin polymer was prepared according to the procedure of Example 11. The procedure of Example 3 was then followed by substituting the glycerin-epichlorohydrin polymer used in Example 3 with the glycol-epichlorohydrin polymer of Example 11. Following the procedure described in Example 3, one mole of dodecyl dimethyl amine was reacted with the glycol-epichlorohydrin polymer to yield the polymeric quaternary ammonium salt product

### EXAMPLE 14

One mole of glycol-epichlorohydrin polymer was prepared according to the procedure of Example 11. The procedure of Example 7 was then followed by substituting the glycerin-epichlorohydrin polymer used in Example 7 with the glycol-epichlorohydrin polymer of Example 11. Following the procedure described in Example 7, one mole (297 g) of a mixed alkyl dimethyl amine (alkyl chains are mixture of C-12, C-14 and C-16) was reacted with the glycol-epichlorohydrin polymer to yield the polymeric quaternary ammonium salt product.

### EXAMPLE 15

One mole of glycol-epichlorohydrin polymer was prepared according to the procedure of Example 11. The procedure of Example 8 was then followed by substituting the glycerin-epichlorohydrin polymer used in Example 8 with the glycol-epichlorohydrin polymer of Example 11. Following the procedure described in Example 8, one mole (166 g) of the mixture of alkyl pyridines was reacted with the glycol-epichlorohydrin polymer to yield the polymeric quaternary ammonium salt product.

### EXAMPLE 16

The polymeric quaternary ammonium salts were tested as corrosion inhibitors using the Rotating Cylinder Electrode (RCE) procedure as known to those having ordinary skill in the art. An Ag/AgCl reference electrode was embedded into a conductive reference bridge gel. A cylindrical coupon was cleaned and weighed and attached to the cylinder holder. Test fluids, a mixture of brine and crude oil, were placed in the testing cell and heated to the test temperature. A potentiostat was connected to the cell and the cylinder was rotated. After the baseline corrosion rate became stable, the polymeric quaternary ammonium salts were added as corrosion inhibitors.

For comparison purposes, a reference product was also tested using this procedure. The reference product was a commercially available phosphate ester-based corrosion inhibitor. The results of the RCE tests using selected polymeric quaternary ammonium salts as synthesized in the Examples above are provided in Table 1. The results of the RCE tests using selected polymeric quaternary ammonium salts as synthesized from selected amines according to the procedures of Example 1 are shown in Table 2. Dosage rates shown in Tables 1 and 2 are based upon the active salt, by volume.

### EXAMPLE 17

The polymeric quaternary ammonium salts were tested as corrosion inhibitors using the corrosion wheel constant concentration test as known to those having ordinary skill in the art and described in a modified form in NACE publication ID182 (December 1982), which is hereby fully incorporated by reference. For comparison purposes, a reference product was also tested using this procedure. The reference product was a commercially available phosphate ester-based corrosion inhibitor.

The results of the wheel tests using selected polymeric quaternary ammonium salts as synthesized in the Examples above are provided in Table 1. The results of the additional wheel tests using selected polymeric quaternary ammonium salts as synthesized from selected amines according to the procedures of Example 1 are shown in Table 2. Dosage rates shown in Tables 1 and 2 are based upon the active salt, by volume.

TABLE 1
Corrosion Inhibitor Performance of Polymeric

| | RCE | Wheel Test | RCE |
|---|---|---|---|
| | 25 ppm dosage | 25 ppm dosage | 15 ppm dosage |
| Inhibitor | wt. loss, mg. | % Protection | % Inhibition |
| Example 4 | 7.3 | ND | 93.0 |
| Example 5 | ND | 87.0 | 86.1 |
| Reference | 8.6 | 85.5 | 97.0 |
| Example 6 | ND | 92.1 | |
| Example 9 | | | 79.0 |
| Example 10 | | | 75.0 |

TABLE 2
Corrosion Inhibitor Performance of Polymeric Salts

| | Wheel Test | RCE |
|---|---|---|
| Amine Used | 7.5 ppm dosage | 7.5 ppm dosage |
| to Form Salt | % inhibition | Corr. Rate, mpy |
| Example 2 | ND | 5.01 |
| Example 5 | ND | 7.9 |
| Exam/ple 7 | 92.1 | ND |
| Example 8 | 50.3 | 4.7 |

### EXAMPLE 18

Using the corrosion wheel constant concentration test as described in Example 17, the polymeric quaternary ammonium salts synthesized in Examples 6 and 8 were tested in a corrosion system consisting of NACE brine and LVT 200 (90:10) that were made sour by bubbling H₂S through the solution. The corrosion test results are shown in Table 3.

TABLE 3
Wheel Test for Sour Conditions

| Inhibitor | Dosage, ppm | % Inhibition |
|---|---|---|
| Example 6 | 15 | 86.0 |
| Example 6 | 25 | 90.0 |
| Example 8 | 15 | 89 |
| Example 8 | 25 | 90 |

### EXAMPLE 19

The effectiveness of the polymeric quaternary ammonium salts as a biocide was tested using the method shown in the API Recommended Practice for Biological Analysis of Subsurface Injection Waters, API RP 38, March, 1982, which is hereby fully incorporated by reference. The effectiveness of the salts as a biocide was tested using planktonic sulfate reducing bacteria (SRB), Aerobic Acid Producing Bacteria (AAPB) and Anaerobic Acid Producing (AnAP) Bacteria.

The desired concentrations of the polymeric quaternary ammonium salt biocide were added to clean sterilized bottles. Two bottles contained no biocides and served as controls. Under a N₂ blanket, 10 mL of actively growing bacterial culture (24 hours old) was added to each of the bottles, and a 1 mL sample was taken and serially diluted into six bottles. The solutions were mixed well and the bottles were placed in an incubator at 37° C. for 4 hours. At the end of this period, 1 mL samples from each of the treated bottles were taken and serially diluted into 3 bottles. Samples from the control bottles were serially diluted into six bottles. All bottles were then placed in an incubator at 37° C. and bacterial growth was monitored for 15 days. The results are shown in Tables 4-6.

TABLE 4
Sulfur Reducing Bacteria

| Chemical | Dosage, ppm | Bacterial cells/ml |
|---|---|---|
| Control | 0 | 10⁵ |
| Glutaraldehyde | 31.3 | 10¹ |
| Glutaraldehyde | 62.5 | 0 |
| Examples 1 | 31.3 | 10¹ |
| Examples 1 | 62.5 | 10¹ |
| Examples 2 | 31.3 | 0 |
| Examples 2 | 62.5 | 0 |
| Examples 3 | 31.3 | 10¹ |
| Examples 3 | 62.5 | 0 |

TABLE 5
Aerobic Acid Producing Bacteria (AAP)

| Chemical* | Dosage, ppm | Bacterial cells/ml |
|---|---|---|
| Control | 0 | ≧10⁶ |
| Glutaraldehyde | 100 | ≧10³ |
| Glutaraldehyde | 200 | ≧10³ |
| Glutaraldehyde | 375 | 10² |
| Glutaraldehyde | 500 | 10¹ |
| Examples 1 | 125 | 10¹ |
| Examples 1 | 250 | 10¹ |
| Examples 1 | 375 | 0 |
| Examples 1 | 500 | 0 |
| Examples 2 | 125 | 0 |
| Examples 2 | 250 | 0 |
| Examples 2 | 375 | 0 |
| Examples 2 | 500 | 0 |
| Examples 3 | 125 | 0 |
| Examples 3 | 250 | 0 |
| Examples 3 | 375 | 0 |
| Examples 3 | 500 | 0 |

TABLE 6
Anaerobic Acid Producing (AnAP) Bacteria

| Chemical | Dosage, ppm | Bacterial cells/ml |
|---|---|---|
| Control | 0 | 10⁵ |
| Glutaraldehyde | 31.3 | 10² |
| Glutaraldehyde | 62.5 | ≧10³ |
| Glutaraldehyde | 125 | ≧10³ |
| Example 1 | 31.3 | 0 |
| Example 1 | 62.5 | 0 |
| Example 1 | 125 | 0 |
| Example 2 | 31.3 | 0 |
| Example 2 | 62.5 | 0 |
| Example 2 | 125 | 0 |
| Example 3 | 31.3 | ≧10³ |
| Example 3 | 62.5 | 0 |
| Example 3 | 125 | 0 |

It will be understood from the foregoing description that various modifications and changes may be made in the preferred embodiment of the present invention without departing from its true spirit. It is intended that this description is for purposes of illustration only and should not be construed in a limiting sense. The scope of this invention should be limited only by the language of the following claims.

1. A composition for use as a biocide and corrosion inhibitor, comprising:
a polymeric quaternary ammonium salt prepared by a reaction of a polyepihalohydrin with a tertiary amine, wherein the polyepihalohydrin is prepared by a polymerization reaction of an epihalohydrin in the presence of a monomeric poly alcohol; and
a solvent carrier.

2. The composition of claim 1, wherein the polymeric quaternary ammonium salt is 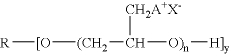
where R is an organic moiety of the poly alcohol n is between 1 and about 10, y is between about 2 and about 150, A is the tertiary amine and X⁻ is a halide.

3. The composition of claim 2, wherein n is between about 3 and about 10.

4. The composition of claim 2, wherein y is between about 6 and about 42.

5. The composition of claim 1, wherein the tertiary amine comprises alkyl functional groups.

6. The composition of claim 1, wherein the tertiary amine comprises a cycloalkyl functional group or an aryl functional group.

7. The composition of claim 1, wherein the tertiary amine is selected from hexadecyl dimethyl amine, tetradecyl dimethyl amine, dodecyl dimethyl amine, imidazoline or alkyl pyridines.

8. The composition of claim 1, wherein the poly alcohol is selected from glycol, glycerin, any tetritols, any pentitols, sorbitol, any hexitols, mannitol, dulcitol, pentaerythritol, dipentaerythritol, and tripentaerythritol.

9. The composition of claim 1, wherein the solvent carrier comprises components selected from water, methanol, isopropyl alcohol or combinations thereof.

10. A method of inhibiting corrosion of metal in contact with a corrosive medium, comprising:
adding a corrosion-inhibiting amount of the composition of claim 1 to the corrosive medium.

11. The method of claim 16, wherein the corrosive medium comprises a petroleum product.

12. The method of claim 11, wherein the petroleum product is crude oil.

13. The method of claim 10 wherein the corrosive medium further comprises a brine.

14. The method of claim 10, wherein the step of adding the composition further comprises:
adding the composition in a batch manner.

15. The method of claim 14, wherein the composition is added at a dosage rate of between about 200 ppm and about 15,000 ppm by volume.

16. The method of claim 10, wherein the step of adding the composition further comprises:
adding the composition in a continuous manner.

17. The method of claim 16, wherein the composition is added at a dosage rate of between about 1 ppm and about 3000 ppm by volume.
